(19) United States
(12) Patent Application Publication
(10) Pub. No.: US 2012/0065546 A1
(43) Pub. Date: Mar. 15, 2012

(54) MEASUREMENT AND ESTIMATION METHOD FOR ORTHOPEDICS DIAGNOSIS

(75) Inventors: Yu-Min WU, New Taipei City (TW); Chih-Hsiung Yu, Taoyuan City (TW); Hsin-Hsueh Wu, Kaohsiung City (TW); Chien-Sheng Wang, Hsinchu City (TW); Tsung-Min Hsieh, Tainan City (TW); Shu-Wei Huang, New Taipei City (TW)

(73) Assignees: INDUSTRIAL TECHNOLOGY RESEARCH INSTITUTE, Hsinchu County (TW); Quanta Computer Inc., Tao Yuan Shien (TW)

(21) Appl. No.: 13/095,181

(22) Filed: Apr. 27, 2011

(30) Foreign Application Priority Data
Sep. 15, 2010 (TW) 099131262

(51) Int. Cl. A61B 5/103

(52) U.S. Cl. 600/587

(57) ABSTRACT

A measurement and estimation method for orthopedics diagnosis is disclosed, which includes providing a resonant frequency of a bone, providing a bone length of the bone, providing a correction parameter, and calculating a bone density according to the resonant frequency, the bone length, and the correction parameter.

### RELATED APPLICATIONS

This application claims priority to Taiwan Application Serial Number 99131262, filed Sep. 15, 2010, which is herein incorporated by reference.

### BACKGROUND

1. Field of Invention

The present invention relates to a measurement and estimation method for orthopedics diagnosis. More particularly, the present invention relates to a method for measuring bone density and estimating artificial hip-joint loosen.

2. Description of Related Art

With rapid development of industry and economy, the life expectancy has become longer and longer. However, when elderly people suffer from Osteoporosis or Osteoarthritis syndrome, ache that occurs from Osteoporosis or Osteoarthritis syndrome tortures the elderly people a lot. Osteoporosis may cause low bone density and raise the risk of fracture. Osteoarthritis syndrome would cause joint ache and joint damage, and the patient may need to do artificial joint replacement if necessary.

In order to get information of people's human bone mineral density, and to diagnose the condition of people's hip-joint (e.g. slack or loose), one of the methods is to send some vibration waves into a human body from a signal output of a vibration source through a target region of the human body. When the vibration waves move in the human body and hit the tested bones, some reflective waves will be generated from the tested bones to move outwards from the human body. Then, the reflective waves will be collected and analyzed to figure out the information of human bone mineral density or the health condition of the hip-joint, so as to further evaluate the health condition of the tested bones and determine a proper medical treatment according to the collected information.

Conventionally, the bone density measurement and the artificial hip-joint estimation are made respectively and cannot be integrated in the same process. Therefore lots of time and cost are spent on bone density measurement and the hip-joint estimation.

For the forgoing reasons, there is a need to improve the efficiency of the bone density measurement and the hip-joint estimation.

### SUMMARY

An object of the invention is to provide a measurement and estimation method for orthopedics diagnosis to improve the efficiency of the bone density measurement and the artificial hip-joint estimation.

An aspect of the invention provides a measurement and estimation method for orthopedics diagnosis, which includes providing resonant frequencies of a bone, providing a bone length of the bone, providing one or more correction parameter, and calculating a bone density according to the resonant frequencies, the bone length, and the correction parameter. The bone can be a femur; the correction parameter is a wrist perimeter, and the bone density is calculated by a following formula: EST BD=CL²f, wherein EST BD is the bone density; C is the wrist perimeter; L is the bone length, and f is the resonant frequency. The correction parameter is a body weight, and the bone density is calculated by a following formula: EST BD=\sqrt{f²wL³}, wherein EST BD is the bone density; w is the body weight; L is the bone length, and f is the resonant frequency. The measurement and estimation method for orthopedics diagnosis further includes placing a vibrating source on a bottom end of the femur and placing a sensor on a top end of the femur for providing the resonant frequency of the femur. The vibrating source provides a sweep frequency vibration, and the sensor detects and provides the resonant frequency. The vibrating source provides a constant frequency vibration, and an amplitude of a fundamental frequency and an amplitude of a harmonic frequency are provided. A hip-joint loosen would be estimated when the amplitude of the harmonic frequency is greater than a predetermined ratio of the amplitude of the fundamental frequency, wherein the predetermined ratio can be 50%.

The method of the present invention places the vibration source and the sensor on the predetermined positions of the subject person and uses the data collected by the sensor for measuring bone density and estimating hip-joint loosen. The present measurement and estimation method for orthopedics diagnosis has the advantages of being noninvasive and nonradioactive, and short estimating time. The method of the present invention further consider the correction parameter, such as the bone length (represented by femur length), the bone perimeter (represented by wrist perimeter), or the bone weight (represented by total body weight), to improve the accuracy of the bone density measurement.

It is to be understood that both the foregoing general description and the following detailed description are by examples, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a correlation diagram of EST BD measured by the present embodiment and by Dual Energy X-ray Absorptiometry (DEXA);

FIG. 2 is a correlation diagram of EST BD measured by the present embodiment and by Dual Energy X-ray Absorptiometry (DEXA);

FIG. 3 and FIG. 4 show the maximum harmonic frequency and the fundamental frequency thereof of different subject people; and

FIG. 5 is a flow chart of an embodiment of the measurement and estimation method for orthopedics diagnosis of the invention.

### DESCRIPTION OF THE EMBODIMENTS

Reference will now be made in detail to the present embodiments of the invention, examples of which are illustrated in the accompanying drawings. Wherever possible, the same reference numbers are used in the drawings and the description to refer to the same or like parts.

The present invention provides a measurement and estimation method for orthopedics diagnosis, which uses a low frequency vibration source as a signal input and a sensor for detecting. The vibration source is placed close to a subject vibration position of a subject person. The subject vibration position can be close to a knee of the subject person, for example, a bottom end of a femur of the subject person. The sensor is placed on a predetermined detect position of the subject person. The sensor can be placed on a bottom of a hip of the subject person. In one embodiment, the sensor can be placed on the top end of the femur of the subject person. More particularly, the sensor is placed close to a great trochanter of the femur of the subject person. The detect result provided by the sensor is utilized for both bone density measurement and hip-joint loosen estimation.

The vibration source would provide a sweep frequency vibration at a low frequency band while measuring the bone density. For example, the vibration source sweeps from 30 Hz to 200 Hz repeatedly, and the sensor can get a resonant frequency of the bone, wherein the resonant frequency can be utilized for measuring the bone density. However, the correlation between the bone density and only the resonant frequency is not really reliable. Therefore the present invention further considers with other correction parameters to improve the accuracy of bone density measurement. The correction parameter can be a length of a part of the body, a perimeter of a part of the body, or a weight of the body.

The vibration source would provide one or more constant frequency vibration at a low frequency band as the input signal while estimating hip-joint loosen. The estimation of hip-joint loosen is made when the sensor detects an obvious harmonic frequency. The measurement and estimation method for orthopedics diagnosis can improve the efficiency and the accuracy of orthopedics diagnosis.

The equation of resonant frequency shown as following.

$${f}_{n} = {α}_{n} \sqrt{\frac{EI}{ρ {AL}^{4}}} \qquad Formula ( 1 )$$

In Formula (1), fₙ is a resonant frequency of a subject; α is a constant; E is Elastic Modulus; I is inertia; ρ is density; A is area; L is length.

According to Formula (1), the density and the shape of the subject would decide the resonant frequency of the subject, wherein the length of the subject would effect the resonant frequency the most. Therefore the applicants found that considering with the body length parameter, especially the bone length, while measuring the bone density would improve the accuracy of bone density measurement. Furthermore, with the placement of the sensor on the hip of the subject person in the present invention, the apparatus can also be used for estimating hip-joint loosen. The femur has the advantage of being close to the skin surface, such that the sensor can get the reflective signal more directly.

According to above description, the vibration source and the sensor are preferably but not limited placed on the positions of opposite ends of the femur. The femur is described in the following description, but other possible bones can also be used in the present invention.

The length of the femur and other possible body parameters can be used as the correction parameter to improve the accuracy of bone density measurement. For example, the correction parameter can be a femur perimeter or a femur weight. Though it is not easy to get the femur perimeter and the femur weight, the value of the femur perimeter and the femur weight can be replaced or calculated by other body length or body weight according to a particular ratio between different parts of body. For example, the femur length can be replaced by a wrist perimeter, and the femur weight can be calculated from total body weight.

First embodiment of the measurement and estimation method for orthopedics diagnosis of the invention is disclosed as following. There is a particular ratio between different parts of human body, therefore perimeter of the femur can be replaced by other body perimeter, which is easier to measure. The length of the femur is represented by the length from the great trochanter to the patella. The resonant frequency of the femur can be provided by using the sweep frequency vibration as described above. The inertia is omitted due to difficult to calculate in a non-uniform subject in Formula (1). The above parameters is substituted in Formula (1), and the Formula (1) can be simplified and becomes Formula (2) as following.

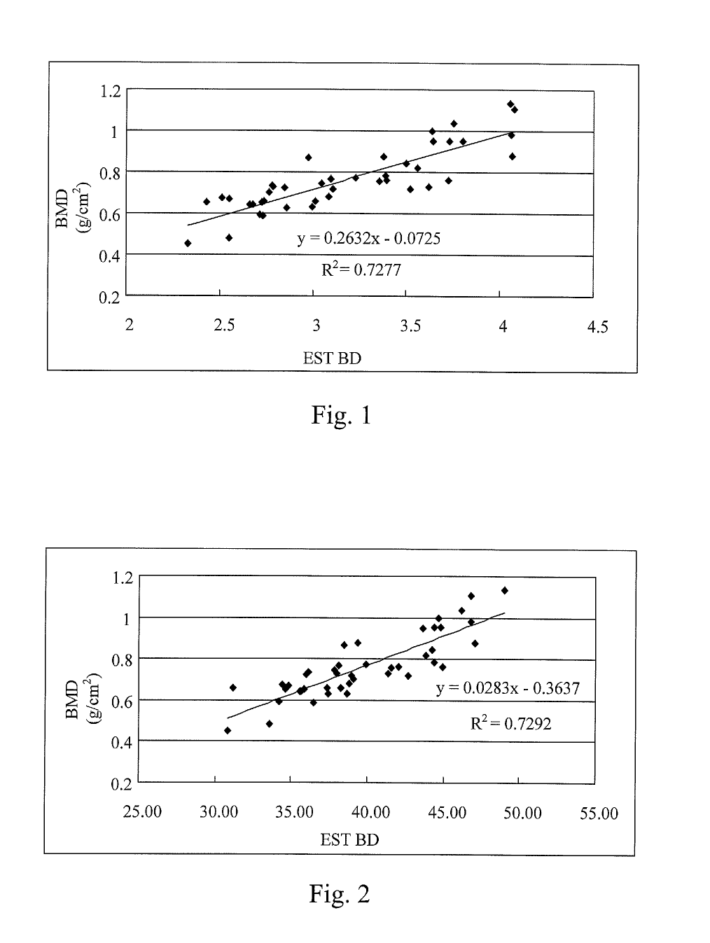

EST BD=CL²f Formula (2)

Wherein EST BD is bone density; C is the wrist perimeter; L is the bone length, and f is the resonant frequency. The comparison between the EST BD values measured by the present embodiment and BMD measured by Dual Energy X-ray Absorptiometry (DEXA) method are shown in FIG. 1.

FIG. 1 is a correlation diagram of EST BD measured by the present embodiment and by DEXA, which is believed as the most accurate method for measuring bone density. X axle shows the EST BD measured by the present embodiment. Y axle shows the BMD measured by DEXA method. In femur density measurement, generally, using DEXA would take about 15-20 minutes, but using the present embodiment would take only 2-3 minutes. The present embodiment would highly shorten the time for measuring bone density, and the correlation coefficient R² of the present embodiment with Dual Energy X-ray Absorptiometry (DEXA) is 0.7277.

Second embodiment of the measurement and estimation method for orthopedics diagnosis of the invention is disclosed as following. The bone weight is related to the body total weight, therefore the bone weight can be replaced by the body total weight with an assumption that the density of the bone is uniform. The above parameters is substituted in Formula (1), and the Formula (1) can be simplified and becomes Formula (3) as following.

EST BD=\sqrt{f²wL³} Formula (3)

Wherein EST BD is the bone density; w is the body total weight; L is the bone length, and f is the resonant frequency. The comparison between EST BD values measured by the present embodiment and BMD measured by Dual Energy X-ray Absorptiometry (DEXA) method are shown in FIG. 2. FIG. 2 is a correlation diagram of EST BD measured by the present embodiment and by DEXA. X axle shows the EST BD measured by the present embodiment. Y axle shows the BMD measured by DEXA method. As shown in FIG. 2, the correlation coefficient R² of the present embodiment with Dual Energy X-ray Absorptiometry (DEXA) is 0.7292.

A constant K₁ or K₂ can be optionally added in Formula (2) or Formula (3), thus the EST BD measured by the present invention can be transfer into BMD.

Third embodiment of the measurement and estimation method for orthopedics diagnosis of the invention is disclosed as following. The present invention can further estimate hip-joint loosen. The hip-joint loosen thereof means the artificial femoral head loosen from the femur. The vibration source can input constant frequency vibration to the femur, and then observe if there is an obvious harmonic frequency output. The estimation of hip-joint loosen would be made when an obvious harmonic frequency is observed.

The resonant frequency of the femur is about 100 Hz. The resonant of soft tissue is less than 50 Hz. The input fundamental frequency is therefore set from 50 Hz to 200 Hz while doing hip-joint loosen test. The hip-joint loosen is estimated when the amplitude of the harmonic frequency is greater than a predetermined ratio, for example 50%, of the amplitude of the fundamental frequency.

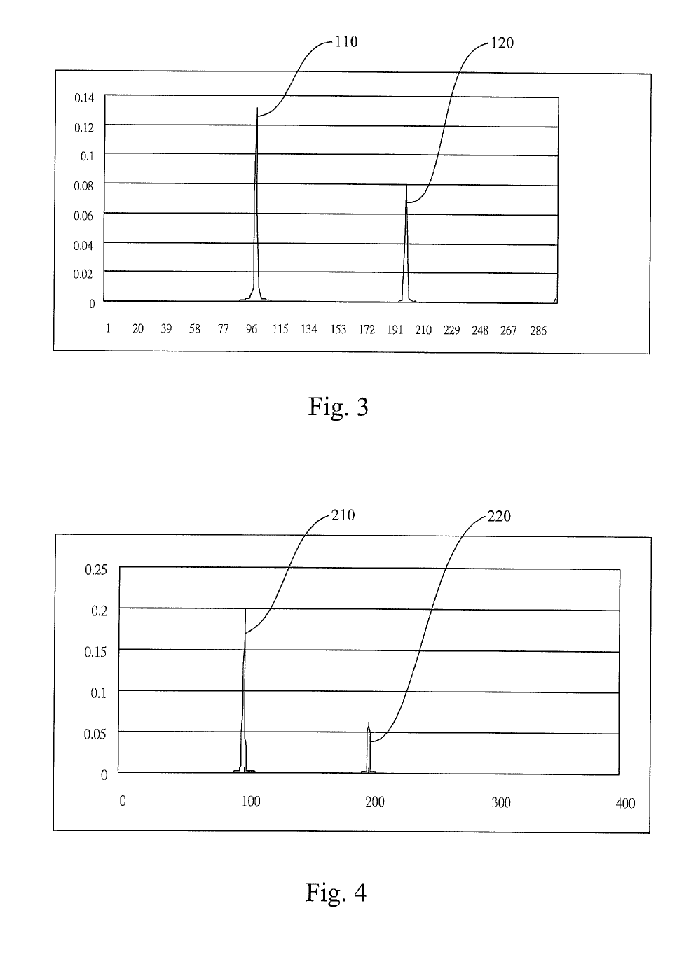

In the present embodiment, the vibration source inputs constant frequency vibrations of 50 Hz, 60 Hz, 70 Hz, 80 Hz, 90 Hz, 100 Hz, 110 Hz, 120 Hz, 130 Hz, 140 Hz, and 150 Hz, wherein each vibration is operated for 10 seconds, and the sensor would detect a maximum harmonic frequency. The maximum harmonic frequency and the fundamental frequency thereof of different subject people are shown in FIG. 3 and FIG. 4.

Refer to FIG. 3. The maximum harmonic frequency 120 is observed when the input fundamental frequency 110 is about 100 Hz. The amplitude of the fundamental frequency 110 is about 0.13 signal magnitude, and the amplitude of the harmonic frequency 120 is about 0.08 signal magnitude. The amplitude of the harmonic frequency 120 is greater than 50% of the amplitude of the fundamental frequency 110, therefore the harmonic frequency 120 is regarded as an obvious harmonic frequency, and an estimation of hip-joint loosen can be made.

Refer to FIG. 4. The maximum harmonic frequency 220 is observed when the input fundamental frequency 210 is about 100 Hz. The amplitude of the fundamental frequency 210 is about 0.2 signal magnitude, and the amplitude of the harmonic frequency 220 is about 0.06 signal magnitude. The amplitude of the harmonic frequency 220 is not greater than 50% of the amplitude of the fundamental frequency 210, therefore the harmonic frequency 220 would not be regarded as an obvious harmonic frequency, and the hip-joint thereof is estimated normal.

As disclosed above, the plural sets of subject people are further analyzed by high-level instrument, and the results analyzed by high-level instrument are the same as the results estimated by the present invention. Therefore the present invention can be used for previously estimating hip-joint loosen and can reduce the screening time.

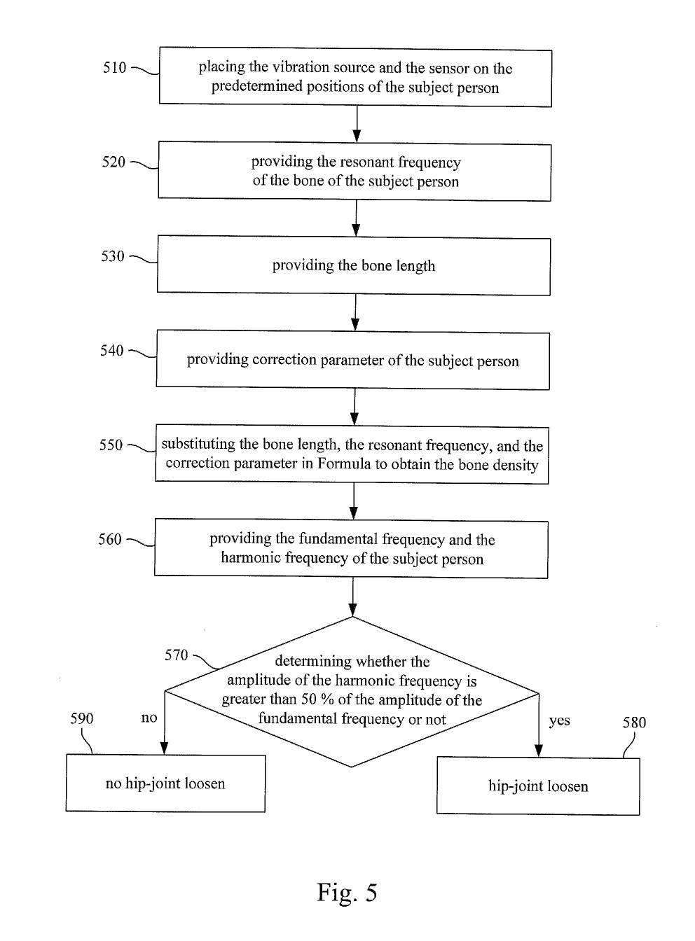

FIG. 5 is a flow chart of an embodiment of the measurement and estimation method for orthopedics diagnosis of the invention. The vibration source and the sensor are placed on the predetermined positions of the subject person in step 510. For example, the vibration source is placed on the bottom end of the femur, and the sensor is placed on the top end of the femur. Then the resonant frequency of the bone of the subject person is provided or detected in Step 520. The bone length is provided in Step 530. The correction parameter of the subject person is provided in Step 540. Then the bone length, the resonant frequency, and the correction parameter are substituted in Formula to obtain the bone density in Step 550. The fundamental frequency and the harmonic frequency of the subject person are provided in Step 560. Then Step 570 is determining whether the amplitude of the harmonic frequency is greater than 50% of the amplitude of the fundamental frequency or not. If the amplitude of the harmonic frequency is greater than 50% of the amplitude of the fundamental frequency, then the estimation of hip-joint loosen is made in Step 580. If the amplitude of the harmonic frequency is not greater than 50% of the amplitude of the fundamental frequency, then the estimation of no hip-joint loosen is made in Step 590.

The vibration source would provide the sweep frequency vibration at a low frequency band for several times in Step 520 to obtain the resonant frequency. The correction parameter in Step 540 can be the perimeter parameter or the weight parameter. The Formula being substituted in Step 550 is EST BD=CL²f when the bone is femur, and the parameter is wrist perimeter, wherein EST BD is bone density; C is the wrist perimeter; L is the bone length, and f is the resonant frequency. The Formula being substituted in Step 550 is EST BD=\sqrt{f²wL³} when the bone is femur, and the parameter is the total body weight, wherein EST BD is the bone density; w is the body total weight; L is the bone length, and f is the resonant frequency. The vibration source inputs constant frequency vibration to the femur in Step 560 at the low frequency band, and the sensor observes the harmonic frequency thereof. The maximum harmonic frequency and the fundamental frequency thereof are compared in Step 570.

According to the above embodiments, the present invention has the following advantages. The method of the present invention places the vibration source and the sensor on the predetermined positions of the subject person and uses the data collected by the sensor for measuring bone density and estimating hip-joint loosen. The present measurement and estimation method for orthopedics diagnosis has the advantages of being noninvasive and nonradioactive, and short estimating time. The method of the present invention further consider the correction parameter, such as the bone length (represented by femur length), the bone perimeter (represented by wrist perimeter), or the bone weight (represented by total body weight), to improve the accuracy of the bone density measurement.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A measurement and estimation method for orthopedics diagnosis, the method comprising:
providing a resonant frequency of a bone;
providing a bone length of the bone;
providing a correction parameter; and
calculating a bone density according to the resonant frequency, the bone length, and the correction parameter.

2. The measurement and estimation method for orthopedics diagnosis of claim 1, wherein the bone is a femur; the correction parameter is a wrist perimeter, and the bone density is calculated by a following formula:
EST BD=CL²f,
is wherein EST BD is the bone density; C is the wrist perimeter; L is the bone length, and f is the resonant frequency.

3. The measurement and estimation method for orthopedics diagnosis of claim 1, wherein the bone is a femur; the correction parameter is a body weight, and the bone density is calculated by a following formula:
EST BD=\sqrt{f²wL³},
wherein EST BD is the bone density; w is the body weight; L is the bone length, and f is the resonant frequency.

4. The measurement and estimation method for orthopedics diagnosis of claim 1, wherein the bone is a femur.

5. The measurement and estimation method for orthopedics diagnosis of claim 4, further comprising placing a vibrating source on a bottom end of the femur and placing a sensor on a top end of the femur for providing the resonant frequency of the femur.

6. The measurement and estimation method for orthopedics diagnosis of claim 5, wherein the vibrating source provides a sweep frequency vibration, and the sensor detects and provides the resonant frequency.

7. The measurement and estimation method for orthopedics diagnosis of claim 5, wherein the vibrating source provides a constant frequency vibration.

8. The measurement and estimation method for orthopedics diagnosis of claim 7, further comprising proving an amplitude of a fundamental frequency and an amplitude of a harmonic frequency.

9. The measurement and estimation method for orthopedics diagnosis of claim 8, wherein hip-joint loosen is estimated when the amplitude of the harmonic frequency is greater than a predetermined ratio of the amplitude of the fundamental frequency.

10. The measurement and estimation method for orthopedics diagnosis of claim 9, wherein the predetermined ratio is 50%.
